(19) United States
(12) Patent Application Publication
(10) Pub. No.: US 2021/0369986 A1
(43) Pub. Date: Dec. 2, 2021

(54) DEVICES AND METHODS OF DELIVERING A GAS TO A WOUND SITE AND AN OPEN SURGICAL SITE

(71) Applicant: Christopher Stephen Blacklock, Chichester (GB)

(72) Inventor: Christopher Stephen Blacklock, Chichester (GB)

(21) Appl. No.: 17/330,486

(22) Filed: May 26, 2021

Related U.S. Application Data
(60) Provisional application No. 63/029,749, filed on May 26, 2020.

(51) Int. Cl. A61M 13/00; B01D 53/22; B01D 53/62

(52) U.S. Cl. CPC A61M 13/00; B01D 53/228; A61M 2209/088; A61M 2202/0225; B01D 2053/221; B01D 53/62

(57) ABSTRACT

Devices and methods for delivering and filtering a gas to a wound site to enhance healing, reduce the potential for bacterial infections, lessen the need for antibiotics and to create a protective and therapeutic gas atmosphere along a treatment site. More particularly, the invention relates to a device arranged to deliver a gas to a treatment site, the device being connectable to a gas source, the device including (i) a first membrane comprising a flexible, microporous polymer body and (ii) a second membrane comprising a flexible nonporous polymer body. Outer edge portions of the second membrane are bonded to outer edge portions of the first membrane to form an interior chamber of the device, the interior chamber of the device confining introduced gas to the interior chamber such that the introduced gas flows out through the pores of the first membrane and is diffused along the treatment site.

### CROSS-REFERENCE TO RELATED APPLICATIONS

The present application claims priority to U.S. Provisional Application Ser. No. 63/029,749 filed on May 26, 2020, which is incorporated herein by reference in its entirety.

### FIELD OF THE INVENTION

The present invention relates to gas diffusion devices, also known as gas insufflators. In particular, this invention relates to a gas diffuser device used to cover a wound and deliver a gas to the wound site to enhance healing and reduce the potential for bacterial infections. The invention also relates to a gas diffuser device used to surround an open surgical site and deliver a gas to the surgical site. The invention also relates to systems and methods for delivering the gas to the gas diffuser device and diffusing the gas through the device into the treatment site.

### BACKGROUND OF THE INVENTION

Wound care is desirable in order to improve health, enhance healing, and to reduce potential for infections of the outer epidermis, as well as underlying dermal and other tissues/organs. Wounds, either injury induced, or surgically induced, such as saphenous vein harvesting, require localized treatment to remedy the affected area and prevent further damage. If wounds are not properly treated, further complications can result, including wound irritation, secondary infections and further discomfort to the subject.

Improper wound care and/or wound healing result in greater costs and expenses, and may require antibiotic use, hospitalizations, and great pain or discomfort to the subject. Present methods of therapeutic wound care may still lead to higher than necessary infection rates and/or longer wound repair/recovery time. Thus, novel therapeutic methods and devices for wound care and wound healing are needed.

Therapeutic gases may be applied to the body for treatment of a variety of medical conditions. Carbon dioxide is a desirable therapeutic gas that has a bacteriostatic function, reducing the growth of bacteria and/or other microorganisms, which possibly may be present on or around medical treatment instruments, and at or around the wound or surgical site. Another desirable therapeutic effect of carbon dioxide gas is its high solubility rate in the tissues of the body relative to oxygen and nitrogen.

During operations which are performed in an open manner, i.e. when an inner portion of the body is uncovered for the performance of the surgical operation, it may be important to prevent air from the environment from reaching the open portion of the body. A gas diffuser (or gas insufflator) can be used to modify the local atmosphere around the operation by delivering the desired gas to the surgical site. Carbon dioxide is heavier than air so that a protective gas atmosphere in a volume adjoining an outwardly open, inner portion of a human being may be created in an easy manner. It is to be noted that the gas may be supplied to the volume in a continuous flow, wherein it is possible to ensure that the surrounding air is prevented from reaching the volume even if a part of the supplied gas leaves the area. During some surgical procedures, gas diffusers/insufflators deliver carbon dioxide gas into the open cavity of the surgical site to modify the local atmosphere in the open cavity so that it is as near to 100 percent CO₂ as possible. This modification of the local atmosphere has been shown to not only reduce the number of air emboli and therefore reduce the potential for a patient to suffer a stroke or organ damage from emboli, but to also have the potential to reduce infections.

Gas diffuser/insufflators are known, but improved gas diffusers that provide a more convenient, inexpensive and accurate delivery of a therapeutic gas to the wound or surgical site while having the ability to maintain a stable local atmosphere of the therapeutic gas are needed. Further needed are gas diffuser/insufflators with improved ability to filter and deliver the therapeutic gas to the treatment site in order to enhance healing, reduce potential for bacterial infection and lessen the need for antibiotics.

### SUMMARY OF THE INVENTION

A gas diffuser device arranged to deliver a gas to a treatment site, the device being connectable to a gas source, the device including (i) a first membrane comprising a flexible, microporous polymer body having a bacterial filtration efficiency of 99.9% or greater and/or having a pore size of 0.2 μm or less and (ii) a second membrane comprising a flexible nonporous or substantially nonporous polymer body. Optionally, the second membrane may comprise a polymer having a pore size smaller than the pore size of the first membrane and/or a bacterial efficiency that is greater than the bacterial efficiency of the first membrane. The first and second membranes of the device each have outer edge portions and the outer edge portions of the second membrane are bonded to the outer edge portions of the first membrane to form an interior chamber of the device, the interior chamber of the device confining introduced gas to the interior chamber such that the introduced gas flows out through the pores of the first membrane. The gas is introduced into the interior chamber through a gas inlet of the device and the gas inlet is connectable to the gas source. The flexible, microporous polymer body of the first membrane of the device is arranged to diffuse the introduced gas into the treatment site and the device is arranged such that the introduced gas maintains a gas atmosphere along the treatment site. The invention also provides a system including a gas source and such a device and a method for delivering a gas to a treatment site.

The invention also provides a gas diffuser device that includes a flexible, microporous polymer body having a bacterial filtration efficiency of 99.0% or greater and/or having a pore size 0.2 μm or greater. The bacterial filtration efficiency of the microporous polymer body could also be 99.5% or greater or could be in a range from 99.0% to 99.9%. Further, the pore size of the microporous polymer body could be in a range of 0.2 μm to 0.4 μm. In embodiments of the invention where the microporous polymer body of the first membrane has a bacterial filtration efficiency of less than 99.9% and/or has a pore size of 0.2 μm or greater, a bacterial filter may be provided in the gas supply line or the gas inlet of the device to filter the gas supply. The invention also provides a system including a gas source and such a device and a method for delivering a gas to a treatment site. It is to be understood that both the foregoing general description and the following detailed description are exemplary and explanatory and are intended to provide further explanation of the invention as claimed.

### BRIEF DESCRIPTION OF THE DRAWINGS

Preferred forms of the present invention will now be described by way of examples with reference to the accompanying drawings.

FIG. 1 shows a device of the invention in cross-section attached to a gas supply line and a gas source.

FIG. 2 shows a view of the wound covering surface of the device.

FIG. 3 shows a view of the non-wound covering surface of the device.

FIG. 4 shows a view of the device of the invention (in cross-section) and system in use for treating a wound.

FIG. 5 shows a view of the device of the invention (in cross-section) and system in use for treating an open surgical site.

FIG. 6 shows and alternate embodiment of the device of the invention.

### DETAILED DESCRIPTION OF THE PREFERRED EMBODIMENTS

In one embodiment the invention is a device arranged to deliver a gas to a treatment site, the device being connectable to a gas source, the device including (i) a first membrane comprising a flexible, microporous polymer body having a bacterial filtration efficiency of 99.9% or greater and (ii) a second membrane comprising a flexible nonporous or substantially nonporous polymer body. Optionally, the second membrane may comprise a polymer having a pore size smaller than the pore size of the first membrane and/or a bacterial efficiency that is greater than the bacterial efficiency of the first membrane. The bacterial filtration efficiency of the flexible, microporous polymer body of the first membrane can be determined/measured in accordance with ASTM F2101-19. The first and second membranes of the device each have outer edge portions and the outer edge portions of the second membrane are bonded to the outer edge portions of the first membrane to form an interior chamber of the device, the interior chamber of the device confining introduced gas to the interior chamber such that the introduced gas flows out through the pores of the first membrane. The gas is introduced into the interior chamber through a gas inlet of the device and the gas inlet is connectable to the gas source. The flexible, microporous polymer body of the first membrane of the device is arranged to diffuse the introduced gas into the treatment site and the device is arranged such that the introduced gas maintains a gas atmosphere along the treatment site.

In another embodiment the invention is a device arranged to deliver a gas to a treatment site, the device being connectable to a gas source, the device including (i) a first membrane comprising a flexible, microporous polymer body having a pore size of 0.2 μm or less and (ii) a second membrane comprising a flexible nonporous or substantially nonporous polymer body. Optionally, the second membrane may comprise a polymer having a pore size smaller than the pore size of the first membrane and/or a bacterial efficiency that is greater than the bacterial efficiency of the first membrane. The first and second membranes of the device each have outer edge portions and the outer edge portions of the second membrane are bonded to the outer edge portions of the first membrane to form an interior chamber of the device, the interior chamber of the device confining introduced gas to the interior chamber such that the introduced gas flows out through the pores of the first membrane. The composition of the material of the second membrane prevents or inhibits the gas from escaping through the second membrane and directs or forces the gas through the pores of the first membrane. The gas is introduced into the interior chamber through a gas inlet of the device and the gas inlet is connectable to the gas source. The flexible, microporous polymer body of the first membrane of the device is arranged to diffuse the introduced gas into the treatment site and the device is arranged such that the introduced gas maintains a gas atmosphere along the treatment site.

The advantage of letting the gas pass through the microporous polymer body of the first membrane having a bacterial filtration efficiency of 99.9% or greater and/or a pore size of 0.2 μm or less is that the pores of the microporous polymer body, which are great in number and positioned very closely to each other, filter bacteria that may present. The average diameter of spherical bacteria is 0.5-2.0 μm. For rod-shaped or filamentous bacteria, the average length is 1-10 μm and the average diameter is 0.25-1.0 μm. Specific examples of bacteria include: E. coli having an average size of about 1.1 to 1.5 μm wide by 2.0 to 6.0 μm long; Spirochetes ranging from 3 to 500 μm in length; the cyanobacterium Oscillatoria being about 7 μm in diameter. Additionally, one group of bacteria called the mycoplasmas, have individuals with size much smaller than these dimensions. They can measure about 0.25 μm and are the smallest cells known so far. They were formerly known as pleuropneumonia-like organisms (PPLO). Mycoplasma gallicepticum, with a size of approximately 200 to 300 nm are thought to be the world smallest bacteria. Therefore a microporous polymer body that has a bacterial filtration efficiency of 99.9% or greater and/or a microporous polymer body having pore sizes of 0.2 μm or less will thus filter and remove most/almost all bacteria from the gas supply. The microporous polymer body of the first membrane having a bacterial filtration efficiency of 99.9% or greater and/or a pore size of 0.2 μm or less can also filter other microorganisms, impurities, or other foreign substances that also may be present.

Another advantage of letting the gas passing through the microporous polymer body of the first membrane is that the pores can function as a multiplicity of supply nozzles, and may distribute the gas in thin layers lying close to each other and forming, when the gas leaves the microporous polymer body, a substantially laminar continuous gas flow. The flexible, microporous polymer body of the first membrane also causes the gas to exit through pores over the majority of the body thereby preventing a singular jetting action.

Diffusion of the gas, preferably carbon dioxide, through the microporous first membrane into the treatment site is advantageous because carbon dioxide has a high solubility in the tissue of the body relative to oxygen and nitrogen, has a bacteriostatic function, which reduces the growth of bacteria and/or other microorganisms and is heavier than air. These features of carbon dioxide aid in the enhancement of healing at the treatment site; aid in the reduction of the potential for bacterial infections at the treatment site; lessen the need for antibiotics at the treatment site; and aids in the creation of a therapeutic and protective gas atmosphere at the treatment site.

In another embodiment the first membrane of the device may include a flexible, microporous polymer body having has a bacterial filtration efficiency of 99.0% or greater, and could be 99.5% or greater. The bacterial filtration efficiency of the flexible, microporous polymer body of the first membrane can be determined/measured in accordance with ASTM F2101-19. In applications where the microporous polymer body of the first membrane has a bacterial filtration efficiency of less than 99.9%, a bacterial filter may be provided in the gas supply line or the gas inlet of the device to filter the gas supply.

In another embodiment the first membrane of the device may include a flexible, microporous polymer body having a pore size of 0.2 μm or greater, and that could be in a range of 0.2 μm to 0.4 μm. In applications where the microporous polymer body of the first membrane has a pore size larger than 0.2 μm, a bacterial filter may be provided in the gas supply line or the gas inlet of the device to filter the gas supply.

In an embodiment the introduced gas is CO₂. In another embodiment the device is arranged to attach to the treatment site by an adhesive positioned on the first membrane, by an adhesive positioned on the second membrane, or both. In an embodiment the device has edges and the device has a shape formed from the edges of the device, the shape being rectangular, square, triangular, oval, trapezoidal or circular In an embodiment the flexible, microporous polymer body of the first membrane is hydrophobic. In an embodiment the flexible, microporous polymer body of the first membrane is made of polytetrafluoroethylene and the flexible, nonporous or substantially nonporous polymer body of the second membrane is also made of a polytetrafluoroethylene. In another embodiment the flexible, microporous polymer body of the first membrane is coated with an antibacterial substance and a medicated substance.

In another embodiment the device includes a pressure relief valve whereby introduced gas in the interior chamber of the device can be diverted from the interior chamber and through the pressure relief valve to maintain a desired gas pressure along the treatment site. In an embodiment the device includes a flush line port whereby introduced gas in the interior chamber of the device can be diverted from the interior chamber and through the flush line port to flush the introduced gas through the device. In another embodiment the device includes an open cell sponge support material, the open cell sponge support material being positioned in the interior chamber in direct contact with the flexible, microporous polymer body of the first membrane. In another embodiment the device includes shapeable structures positioned throughout the device and arranged to conform and shape the device around the treatment site.

The invention also provides a system comprising a gas source and such a device having a first membrane with a microporous polymer body having a bacterial filtration efficiency of 99.9% or greater and/or a pore size of 0.2 μm or less. The gas insert of the device can be connected directly to the gas source or can be connected to a flexible hose portion (or other gas delivery means) that is then connected to the gas supply. In some embodiments of the system, the microporous polymer body of the first membrane of the device may have a bacterial filtration efficiency of less than 99.9% and/or a pore size larger than 0.2 μm. In such embodiments of the system, a filter may be provided in the flexible hose portion (or other gas delivery means) or the gas inlet of the device to filter the gas supply. The filter positioned in the gas inlet or the flexible hose portion may have a housing made of polypropylene and may have glass fibers as a filter material. The filter may have a pore size between 0.1 to 0.4 μm. It is to be understood that the filter could be made of any desired material and could have any desired pore size as needed to properly and adequately filter the gas supply. In an embodiment, the gas comprises a majority of carbon dioxide.

The invention provides a method for delivering a gas to a treatment site including providing a device arranged to deliver gas to a treatment site, the device being connectable to a gas source, the device having (i) a first membrane comprising a flexible, microporous polymer body having a bacterial filtration efficiency of 99.9% or greater and/or a pore size of 0.2 μm or less and (ii) a second membrane comprising a flexible nonporous or substantially nonporous polymer body. Optionally, the second membrane may comprise a polymer having a pore size smaller than the pore size of the first membrane and/or a bacterial efficiency that is greater than the bacterial efficiency of the first membrane. The first and second membranes of the device each have outer edge portions and the outer edge portions of the second membrane are bonded to the outer edge portions of the first membrane to form an interior chamber of the device. The composition of the material of the second membrane prevents or inhibits the gas from escaping through the second membrane and directs or forces the gas through the pores of the first membrane. The method including positioning and attaching the device to the treatment site; connecting the gas inlet of the device to the gas source; and supplying the gas to the interior chamber of the device through the gas inlet, the interior chamber of the device confining the supplied gas such that the supplied gas flows out through the pores of the first membrane of the device and diffuses the supplied gas into the treatment site. The method includes that the supplied gas maintains a gas atmosphere along the treatment site.

In some embodiments of the method, the microporous polymer body of the first membrane of the device may have a bacterial filtration efficiency of less than 99.9% and/or a pore size larger than 0.2 μm. In such embodiments, the first membrane could have a bacterial filtration efficiency of 99.0% or greater, 99.5% or greater or could be in a range of 99.0% to 99.9%. Additionally/alternatively the first membrane could have a pore size in a range of 0.2 μm to 0.4 μm. In such embodiments of the method, a bacterial filter may be provided in the flexible hose portion of the gas source or the gas inlet of the device to filter the gas supply. In an embodiment, the gas comprises a majority of carbon dioxide.

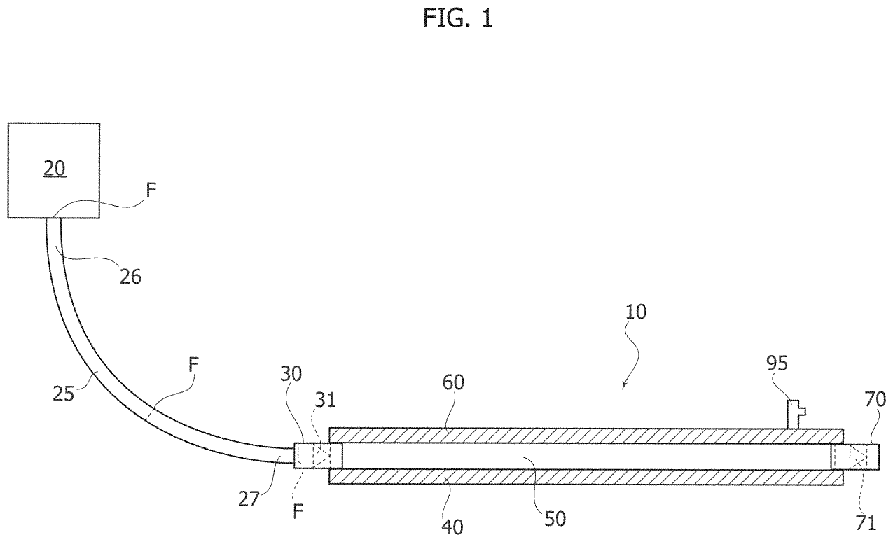

FIG. 1 shows a gas diffuser or insufflator device 10 of the invention (in cross-section connected to a gas source 20. The gas diffuser device 10 has gas inlet 30, first membrane 40, interior chamber 50, second membrane 60 and flush line outlet or port 70 (optional). Gas diffuser device 10 may also have an overpressure relief valve 95. Gas may be supplied from the gas source to the device by flexible gas hose 25 that has intake end 26 connected to gas source 20 and discharge end 27 connected to the gas inlet 30 of the gas diffuser device 10. It should be understood that any desired gas delivery structure or means can be used to supply gas to the device.

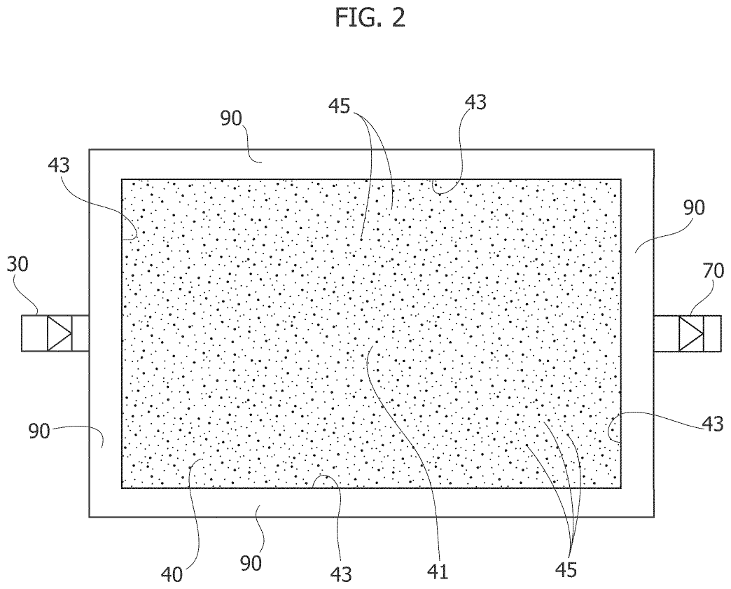

FIG. 2 shows a view of the wound covering surface of gas diffuser 10. First membrane 40 is a microporous membrane through which a supplied gas is filtered and diffused to a treatment site. The treatment site may be a wound or surgical site depending upon the application. First membrane 40 may be made of a flexible microporous polymer such as polytetrafluoroethylene and, more specifically, could be made of POREX® Virtek™ PTFE MD10. It is to be understood that the material composition of first membrane 40 is non-limiting and could be any microporous polymer that filters bacteria and other impurities as discussed further below. First membrane 40 may have a thickness as desired and could be 0.13 mm. First membrane 40 may be hydrophobic and non-wetting. First membrane 40 has pores 45 positioned close together across the first membrane 40 and are distributed across the first membrane in great number and high concentration. Pores 45 prevent the passage of bacteria and other microbes in addition to other impurities larger than the size of the pore from diffusing through first membrane 40 to the treatment site, thereby filtering the gas supplied or introduced to the device. Thus, first membrane 40 can function as a bacterial filter allowing the desired gas to pass through pores 45 while preventing bacteria from passing through pores 45. The bacterial filtration property of first membrane 40 can have a desired efficiency of bacterial filtration. The bacterial filtration efficiency of the flexible, microporous polymer body of the first membrane 40 can be determined/measured in accordance with ASTM F2101-19. The desired bacterial efficiency of first membrane 40 as determined by ASTM F2102-19 could be 99.9% or greater, 99.0% or greater, 99.5% or greater or could be in a range of 99.0% to 99.9% as desired. (It should be understood that the bacterial filtration efficiency could also be less than 99.0% depending upon the application). In some applications where the bacterial filtration efficiency of first membrane 40 is less than 99.9%, a separate filter (F shown with optional locations in FIG. 1) may be provided to the gas source, gas supply line or the gas inlet of the gas diffuser device to filter out bacteria and other impurities.

Optionally, pores 45 of first membrane 40 may have a specific desired size. The desired size could be 0.2 microns or less, the desired pore size could be 0.2 microns or greater, or the desired pore size could be in a range of 0.2 μm to 0.4 μm. Pores 45 that are sized 0.2 microns or less can function as a bacterial filter since the average diameter of spherical bacteria is 0.5-2.0 μm, and for rod-shaped or filamentous bacteria, the average length is 1-10 μm and the average diameter is 0.25-1.0 μm. In some applications, the first membrane 40 will have pores 45 sized larger than 0.2 microns and, in those circumstances, a separate filter (F shown with optional locations in FIG. 1) may be provided to the gas source, gas supply line or the gas inlet of the gas diffuser device to filter out bacteria and other impurities.

First membrane 40 may have body portion 41 surrounded by edge portions 43. Body portion 41 and edge portions 43 may all have pores 45. The gas diffusion device 10 can include adhesive surfaces 90, which may be covered by release paper. Adhesive surfaces 90 adhere to the patient's body surrounding the treatment site. Adhesive surfaces 90 could be an adhesive coating applied to the device or could be a separate film that is attached to or covers the device.

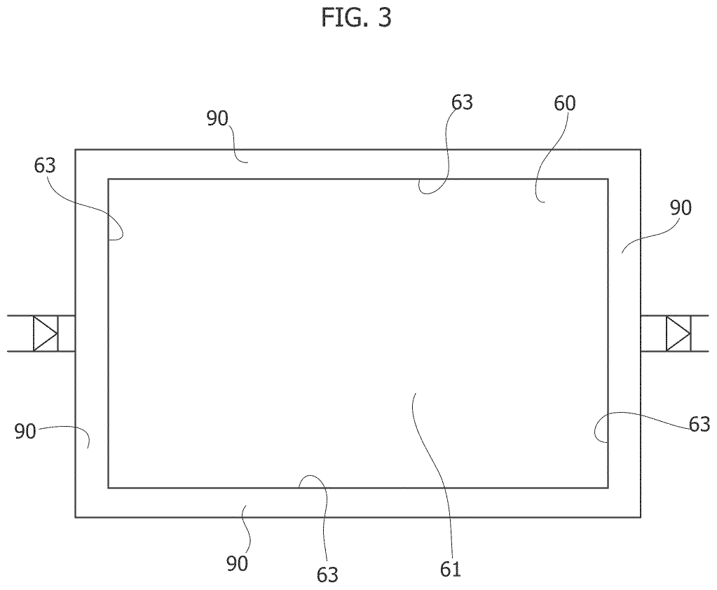

As seen in FIGS. 1 and 3, second membrane 60 may be made of any desired material and could be made from a polymer, a thin metal foil, surgical steel, leather and/or any material that will hold its structural integrity during use and is capable of being sterilized. Second membrane 60 may be flexible or in some embodiments may not be flexible as to hold its shape during usage. The second membrane could also be made out of the same polymer as the first membrane 40, such as a polytetrafluoroethylene and, more specifically, could be made of POREX® Virtek™ PTFE MD10. The second membrane may have any thickness as desired and could be 0.13 mm, and may also be hydrophobic and non-wetting. Second membrane 60 may be nonporous or substantially nonporous. The second membrane 60 may have a pore size smaller than the pore sizes (in some applications much smaller) of first membrane 40 and/or a bacterial efficiency rate that is greater than the bacterial efficiency rate of first membrane 40, thereby directing the flow of gas through gas diffuser 10 and through first membrane 40 while preventing or decreasing (in some applications greatly decreasing) the escape or diffusion of gas through the second membrane. As such, the smaller pore size and/or the greater bacterial efficiency rate of second membrane 60 allows for the second membrane to be nonporous or substantially nonporous, as compared to first membrane 40. In an optional embodiment, second membrane 60 may be made of a number of sheets or layers of a porous material that are bonded/adhered/affixed to one another to make the second membrane 60 less porous or substantially nonporous. In an alternative embodiment, second membrane may be made from the same material as first membrane 40 with a backing sheet affixed to it for stopping the flow of gas through second membrane 60 until such a time that the wound is required to breath or be observed. At this time the backing sheet can be removed and the wound can breath, be allowed to dry and be observed.

Second membrane 60 may have body portion 61 surrounded by edge portions 63. Edge portions 63 of second membrane 60 are bonded/secured/adhered to edge portions 43 of first membrane by sonic welding, thermal welding, compression sealing, induction heating, adhesive or other processes known in the art. The bonding of outer edge portions 63 of the second membrane 60 to the outer edge portions 43 of the first membrane 40 form interior chamber 50 of the device. Second membrane 60 can prevent or inhibit gas introduced into the interior chamber from escaping. The second membrane 60 confines/directs the flow of the introduced gas in interior chamber 50 such that the introduced gas flows out or diffuses through the pores 45 of the first membrane 40 to the treatment site. Some sections of outer edge portions 63 of second membrane 60 and outer edge portions 43 of first membrane 40 are bonded/secured/adhered to portions of gas inlet 30 and optional flush line outlet or port 70. The device 10 may include adhesive surfaces 90, which may be covered by release paper and adhere to the patient's body surrounding the treatment site. Adhesive surfaces 90 can be an adhesive coating applied to the device or to either/both of the first and second membranes. Alternatively, adhesive surfaces 90 can also be a separate film that is attached to the device or to either/both of the first and second membranes.

Gas inlet 30 of device 10 can connect to the flexible gas hose (or other gas delivery means) and to the gas source through various means as known in the art. For example, a barbed or smooth push fitting could be used to connect the gas inlet to the gas supply hose for delivery of CO2 when it is deemed that the connection to the supply of gas should be connected for longer term care. Additionally, there is also the option of having quick disconnect fittings and screw fittings for supplying smaller durations of CO2 to the device that allow for easier/quicker disconnection of the gas delivery means. In order to maintain the therapeutic gas atmosphere along the treatment site and inside the device, the gas inlet may have a one way valve 31 to prevent gas that enters the device through gas inlet 30 from escaping back through the gas inlet. Alternatively/additionally, a Halkey-Roberts clamp, a plug, a cap or a tap could be used to prevent gas from escaping the device back through the gas inlet.

Optional flush line outlet or port 70 may be provided so that the gas delivered to the gas diffusion device can be flushed through the device, thereby removing gases/air, condensation, fluids or impurities that may exist at the treatment site and that may have built up within the device during use. The flush line outlet 70 may have a one way valve 71 to allow gas within the device to have a controlled exit or release from the device through the flush line outlet. The one way valve prevents any uncontrolled gas/air that is outside the device from entering into the device. Alternatively/additionally, a Halkey-Roberts clamp, a plug, a cap or a tap could be used to prevent gas from entering the device through the flush line outlet. The flush line outlet may have an option of having a three way tap on it. The open end of the purge line may have a female luer connection to allow a syringe to be attached when required to draw off any gas or fluid as required from the device and treatment site.

Gas diffuser device 10 may also have an overpressure relief valve 95 (optional) attached thereto allowing gas to be released or purged from the device when the pressure inside the device becomes too high and passes a desired threshold, thereby reducing the risk of damaging the device and potentially the treatment site.

Pore size of the flexible microporous polymer membrane can be determined using the Mercury Intrusion Method. In a vacuum, a mercury drop will not enter a pore due to its very high surface tension, but will if pressure is applied. It is known that, for a given pore size, a certain pressure is required to force the mercury into the pore. For each incremental increase in pressure, the change in intrusion volume is equal to the volume of the pores whose diameters fall within an interval that corresponds to the particular pressure interval. The amount of displaced mercury can therefore be used to calculate the pore size using a graphical representation. The pore size will be the average size of the pore distribution obtained (i.e. the peak value).

The Washburn Equation can be used to convert pressure to pore diameter:

D=−4y(cos θ)/P

where D=Diameter of pore being intruded
y=Surface tension of mercury
P=Intrusion pressure
θ=contact angle between mercury & material

For example, to arrive at a pore size in μm, y is 480 N/m, θ is in degrees, and P is the intrusion pressure, the pressure at which 50% of the volume of mercury intrudes into the pores. The cumulative volume starts at zero and pressure is applied until no more mercury can be introduced (giving total volume of the pores at this point). In a typical test, a graph of cumulative volume (mm³/g) versus intrusion pressure (kPa) is made. The intrusion pressure is then read off the graph. This is the “50% value”. It means that 50% of the pores lie above this diameter and 50% lie below it. Pore size in this application, including the claims, means this 50% value, with 50% of the pores being above this diameter and 50% being below it. The pores 45 of the microporous flexible polymer first membrane 40 allow the gas, preferably CO₂, to diffuse over the full surface area of the membrane. The small pore size means that even at flows as low at 2.5 liters per minute (LPM) it will still act as a very efficient gas diffuser. The smaller pore size means in effect that the gas has to make more effort to exit the microporous flexible polymer first membrane 40 thereby flowing through more pores 45. As such, an almost instantaneous therapeutic gas atmosphere can be formed along the treatment site once the gas is introduced through positive pressure to the gas diffusion device 10. The introduced gas within the interior chamber of the gas diffusion device can have a higher velocity than the gas along the treatment site that has been diffused through the pores 45 of first membrane 40. Thus the flow/diffusion of the introduced gas through the pores of the first membrane can create a slow, substantially laminar, gas flow whereby turbulence of the gas atmosphere is minimized.

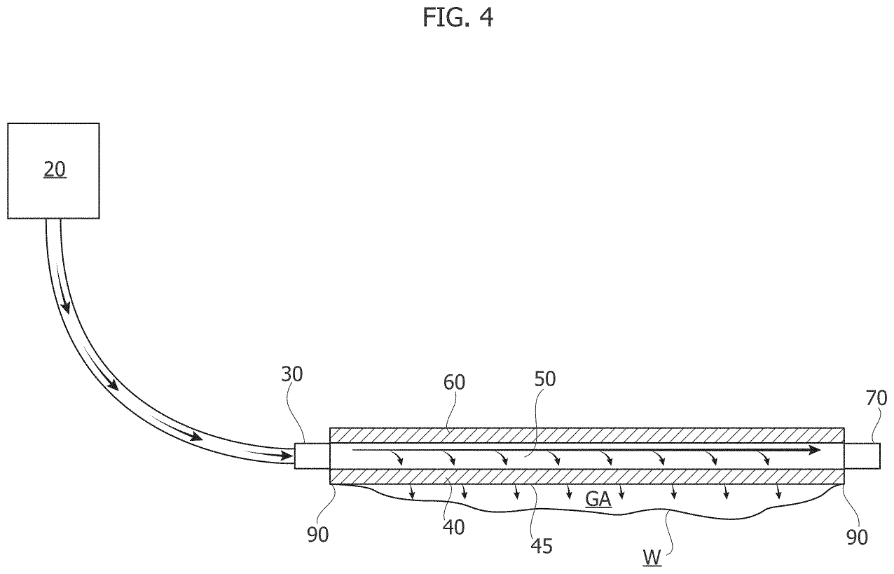

FIG. 4 shows gas diffusion device 10 (in cross-section) in use for treating a wound W. Adhesion surfaces 90 are secured/affixed to the patient's body such that the device covers and surrounds the wound to be treated. Gas flows via positive pressure from gas source 20 through flexible hose 25, through gas inlet 30, and into interior chamber 50 of the gas diffusion device 10 (gas flow and diffusion across first membrane 40 shown with directional arrows). The gas, which may be carbon dioxide, flows out through the multiplicity of pores 45 of flexible, microporous polymer first membrane 40 (pores 45 are indicated in FIG. 2 but are too small to actually be seen) into the treatment site of the wound creating a gas atmosphere GA along the treatment site and wound. The gas may flow at any desired rate as to allow proper and adequate diffusion along the treatment site and could, for example, have a flow rate of greater than 0.0001 l/hr/cm² Δp 70 mbar. In some embodiments, the gas (and other impurities) can be flushed out from the device through optional flush line outlet 70. In some embodiments gas delivered to the device having a pressure higher than a desired threshold can be purged or released through optional overpressure valve 95, thereby protecting the device and the wound from damage.

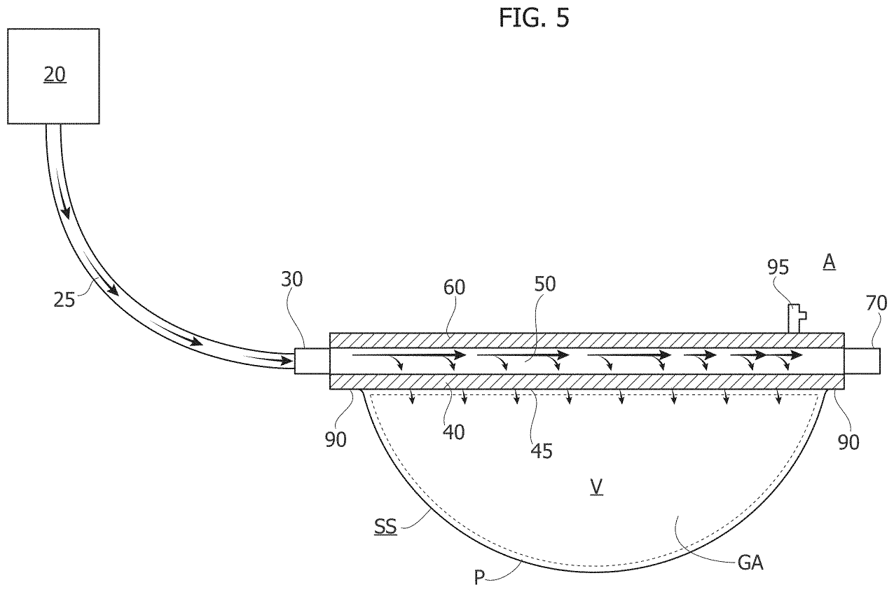

FIG. 5 shows gas diffusion device 10 in use for treating an open surgical site SS. Adhesion surfaces 90 are secured to the patient's body around open volume V and adjacent to a portion P of a human body that is normally not exposed to the atmosphere, as in a surgery. Gas flows via positive pressure from gas source 20 through flexible hose 25, through gas inlet 30, into interior chamber 50 of the gas diffusion device 10 (gas flow and diffusion across first membrane 40 shown with directional arrows). The gas, preferably carbon dioxide, flows out through the multiplicity of pores 45 of flexible, microporous polymer first membrane 40 (pores 45 are indicated in FIG. 2 but are too small to actually be seen) which fills the volume V forming a protective therapeutic gas atmosphere GA and preventing air A from the environment from reaching the volume. As CO2, the preferred gas, is heavier than air, the CO2 will accumulate in the volume V as long as the gas flow into the volume V is not turbulent. In some embodiments, the gas (and other impurities) can be flushed out from the device through optional flush line outlet 70. In some embodiments gas delivered to the device having a pressure higher than a desired threshold can be purged or released through optional overpressure valve 95, thereby protecting the device and the wound from damage.

In order to prevent air embolism, i.e., a blocking of the capillaries and small vessels which may be caused by an air bubble, the therapeutic gas atmosphere in a volume adjoining a temporarily, outwardly open portion of a human being ought to include a delivered gas, the majority of the gas being carbon dioxide. In the applications where a therapeutic gas atmosphere is to be created in a volume adjoining an outwardly open inner portion of the body of a human being or an animal, it is advantageous that the gas includes carbon dioxide due to the fact that carbon dioxide has a high solubility in the tissue of the body relative to oxygen and nitrogen and because carbon dioxide is heavier than air. It is to be noted that the gas may be supplied to the volume in a continuous flow, wherein it is possible to ensure that the surrounding air is prevented from reaching the volume even if a part of the supplied gas leaves the area. Another possibility is, at least initially, to supply gas continuously in order to create therapeutic gas atmosphere, and then supply gas periodically to maintain the gas atmosphere. It should also be noted that the gas may include oxygen, for instance in the cases when said tissue of said open body portion is strongly oxygen dependent. Oxygen, as well as carbon dioxide, is heavier than air so that the protecting atmosphere in the volume may be created in an easy manner since the heavier gas will pass downwardly in the open body portion and force away the non-sterile air present in the lower part of this open portion. In certain applications a protecting atmosphere including sterile air may be satisfactory. The main thing is that air from the environment, i.e., non-sterile air, is prevented from reaching the volume.

In one embodiment, the gas diffusion device will include a flexible shapeable gas delivery tube or hose extending from the gas inlet through the interior chamber of the gas diffusion device. The flexible, shapeable gas delivery tube includes multiple perforations or pores. The shapeable tube will allow the gas diffusion device to be shaped so that in certain instances it can surround the surgical site.

In one embodiment, the gas diffusion device will include shapeable structures within the first and second membranes and interior chamber which will allow the gas diffusion device to be molded around a surgical site in order to create the gas atmosphere.

In one embodiment, the microporous first membrane can also be coated in an antibacterial substance and also a medicated substance to aid healing and reduce pain and infections.

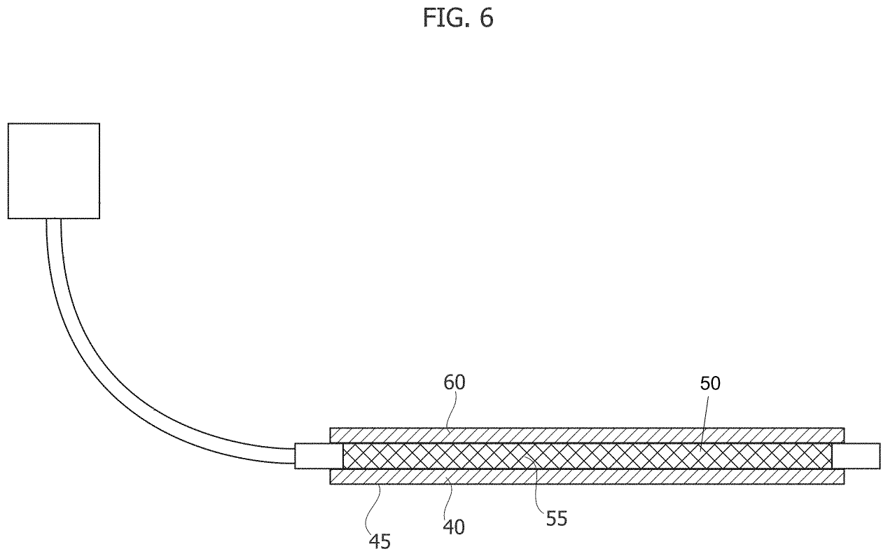

In one embodiment shown in FIG. 6, the gas diffusion device will include an open cell sponge support material 55 positioned along the first membrane inside the interior chamber 50 of the device. The open cell sponge support material will aid the CO₂ gas in diffusing along the majority of the first membrane 40 and through pores 45. In another embodiment the support material inside the interior chamber could have baking soda or another similar chemical compound impregnated into the support material 55 between the first and second membrane. An acidic fluid such as vinegar, rather than a gas could be supplied through the inlet of the device. The acidic fluid in contact with the baking soda or other similar compound causes a chemical reaction whereby carbon dioxide is created. The carbon dioxide is then diffused to the treatment site through pores 45 located in the microporous first membrane 40.

Although particular embodiments have been disclosed herein in detail, this has been done for purposes of illustration only, and is not intended to be limiting with respect to the scope of the following appended claims. In particular, it is contemplated by the inventors that various substitutions, alterations, and modifications may be made to the invention without departing from the spirit and scope of the invention as defined by the claims.

### SPECIFIC EMBODIMENTS

The present disclosure is exemplified by the specific embodiments below.

1. A device arranged to deliver a gas to a treatment site, the device being connectable to a gas source, the device comprising: (i) a first membrane comprising a flexible, microporous polymer body having a bacterial filtration efficiency of 99.9% or greater and (ii) a second membrane comprising a flexible polymer body, wherein the first and second membranes each have outer edge portions and the outer edge portions of the second membrane are bonded to the outer edge portions of the first membrane to form an interior chamber of the device, the interior chamber of the device confining introduced gas to the interior chamber such that the introduced gas flows out through pores of the first membrane, the gas being introduced into the chamber through a gas inlet of the device, the gas inlet being connectable to the gas source, wherein the flexible, microporous polymer body of the first membrane is arranged to diffuse the introduced gas into the treatment site and wherein the introduced gas maintains a gas atmosphere along the treatment site.

2. The device of embodiment 1, wherein the bacterial filtration efficiency of 99.9% or greater of the flexible, microporous polymer body of the first membrane is as determined in accordance with ASTM F2101-19.

3. The device of embodiment 2, wherein the introduced gas is CO₂.

4. The device of embodiment 2, wherein the device is arranged to attach to the treatment site.

5. The device of embodiment 4, wherein the device is attached to the treatment site by an adhesive positioned on the first membrane.

6. The device of embodiment 4, wherein the device is attached to the treatment site by an adhesive positioned on the second membrane.

7. The device of embodiment 2, wherein the device has edges and wherein the device has a shape formed from the edges of the device, the shape being rectangular, square, triangular, oval, trapezoidal or circular.

8. The device of embodiment 2, wherein the flexible, microporous polymer body of the first membrane is hydrophobic.

9. The device of embodiment 2, wherein the flexible, microporous polymer body of the first membrane is made of polytetrafluoroethylene.

10. The device of embodiment 9, wherein the flexible polymer body of the second membrane is nonporous.

11. The device of embodiment 2, further comprising a pressure relief valve and wherein introduced gas in the interior chamber of the device can be diverted from the interior chamber and through the pressure relief valve to maintain a desired gas pressure along the treatment site.

12. The device of embodiment 2, further comprising a flush line port and wherein introduced gas in the interior chamber of the device can be diverted from the interior chamber and through the flush line port to flush the introduced gas through the device.

13. The device of embodiment 2, wherein the flexible, microporous polymer body of the first membrane is coated with an antibacterial substance and a medicated substance.

14. The device of embodiment 2, further comprising an open cell sponge support material, the open cell sponge support material being positioned in the interior chamber in direct contact with the flexible, microporous polymer body of the first membrane.

15. The device of embodiment 2, further comprising shapeable structures positioned throughout the device and arranged to conform the device around the treatment site.

16. The device of embodiment 2, wherein the treatment site is a wound.

17. The device of embodiment 2, wherein the treatment site is a surgical site.

18. A device arranged to deliver a gas to a treatment site, the device being connectable to a gas source, the device comprising: (i) a first membrane comprising a flexible, microporous polymer body having a bacterial filtration efficiency of 99.0% or greater and (ii) a second membrane comprising a flexible polymer body, wherein the first and second membranes each have outer edge portions and the outer edge portions of the second membrane are bonded to the outer edge portions of the first membrane to form an interior chamber of the device, the interior chamber of the device confining introduced gas to the interior chamber such that the introduced gas can only flow out through pores of the first membrane, the gas being introduced into the chamber through a gas inlet of the device, the gas inlet being connectable to the gas source, wherein the flexible, microporous polymer body of the first membrane is arranged to diffuse the introduced gas into the treatment site and wherein the introduced gas maintains a gas atmosphere along the treatment site.

19. The device of embodiment 18, wherein the bacterial filtration efficiency of 99.0% or greater of the flexible, microporous polymer body of the first membrane is as determined in accordance with ASTM F2101-19.

20. The device of embodiment 19, wherein the gas inlet has a bacterial filter.

21. The device of embodiment 19, wherein the flexible, microporous polymer body of the first membrane has a bacterial filtration efficiency of 99.5% or greater

22. The device of embodiment 21, wherein the gas inlet has a bacterial filter.

23. The device of embodiment 20, wherein the introduced gas is CO₂.

24. The device of embodiment 20, wherein the device is arranged to adhere to the treatment site.

25. The device of embodiment 24, wherein the device is attached to the treatment site by adhesive surfaces attached to the device.

26. The device of embodiment 20, wherein the microporous polymer body of the first membrane has a bacterial filtration efficiency in the range of 99.0% to 99.9%.

27. The device of embodiment 20, further comprising a pressure relief valve and wherein introduced gas in the interior chamber of the device can be diverted from the interior chamber and through the pressure relief valve to maintain a desired gas pressure along the treatment site.

28. The device of embodiment 20, further comprising a flush line port and wherein introduced gas in the interior chamber of the device can be diverted from the interior chamber and through the flush line port to flush the introduced gas through the device.

29. The device of embodiment 20, wherein the flexible, microporous polymer body of the first membrane is coated with an antibacterial substance and a medicated substance.

30. The device of embodiment 20, further comprising an open cell sponge support material, the open cell sponge support material being positioned in the interior chamber in direct contact with the flexible, microporous polymer body of the first membrane.

31. The device of embodiment 20, further comprising shapeable structures positioned throughout the device and arranged to conform the device around the treatment site.

32. A system comprising a gas source and a device, the device being arranged to deliver a gas to a treatment site, the device being connectable to a gas source, the device comprising: (i) a first membrane comprising a flexible, microporous polymer body having a bacterial filtration efficiency of 99.9% or greater and (ii) a second membrane comprising a flexible polymer body, wherein the first and second membranes each have outer edge portions and the outer edge portions of the second membrane are bonded to the outer edge portions of the first membrane to form an interior chamber of the device, the interior chamber of the device confining introduced gas to the interior chamber such that the introduced gas flows out through pores of the first membrane, the gas being introduced into the chamber through a gas inlet of the device, the gas inlet being connectable to the gas source, wherein the flexible, microporous polymer body of the first membrane is arranged to diffuse the introduced gas into the treatment site and wherein the introduced gas maintains a gas atmosphere along the treatment site.

33. The system of embodiment 32, wherein the bacterial filtration efficiency of 99.9% or greater of the flexible, microporous polymer body of the first membrane of the device is as determined in accordance with ASTM F2101-19.

34. The system of embodiment 33, wherein the introduced gas is CO₂.

35. The system of embodiment 33, wherein the device is arranged to attach to the treatment site.

36. The system of embodiment 35, wherein the device is attached to the treatment site by an adhesive positioned on the first membrane.

37. The system of embodiment 35, wherein the device is attached to the treatment site by an adhesive positioned on the second membrane.

38. The system of embodiment 33 wherein the device has edges and wherein the device has a shape formed from the edges of the device, the shape being rectangular, square, triangular, oval, trapezoidal or circular.

39. The system of embodiment 33, wherein the flexible, microporous polymer body of the first membrane is hydrophobic.

40. The system of embodiment 33, wherein the flexible, microporous polymer body of the first membrane is made of polytetrafluoroethylene.

41. The system of embodiment 40, wherein the flexible polymer body of the second membrane is made of polytetrafluoroethylene.

42. The system of embodiment 33, further comprising a pressure relief valve and wherein introduced gas in the interior chamber of the device can be diverted from the interior chamber and through the pressure relief valve to maintain a desired gas pressure along the treatment site.

43. The system of embodiment 33, further comprising a flush line port and wherein introduced gas in the interior chamber of the device can be diverted from the interior chamber and through the flush line port to flush the introduced gas through the device.

44. The system of embodiment 33, wherein the flexible, microporous polymer body of the first membrane is coated with an antibacterial substance and a medicated substance.

45. The system of embodiment 33, further comprising an open cell sponge support material, the open cell sponge support material being positioned in the interior chamber in direct contact with the flexible, microporous polymer body of the first membrane.

46. The system of embodiment 33, further comprising shapeable structures positioned throughout the device and arranged to conform the device around the treatment site.

47. A system comprising a gas source and a device, the device being arranged to deliver a gas to a treatment site, the device being connectable to a gas source, the device comprising: (i) a first membrane comprising a flexible, microporous polymer body having a bacterial filtration efficiency of 99.0% or greater and (ii) a second membrane comprising a flexible polymer body, wherein the first and second membranes each have outer edge portions and the outer edge portions of the second membrane are bonded to the outer edge portions of the first membrane to form an interior chamber of the device, the interior chamber of the device confining introduced gas to the interior chamber such that the introduced gas flows out through the pores of the first membrane, the gas being introduced into the chamber through a gas inlet of the device, the gas inlet being connectable to the gas source, wherein the flexible, microporous polymer body of the first membrane is arranged to diffuse the introduced gas into the treatment site and wherein the introduced gas maintains a gas atmosphere along the treatment site.

48. The system of embodiment 47, wherein the bacterial filtration efficiency of 99.0% or greater of the flexible, microporous polymer body of the first membrane of the device is as determined in accordance with ASTM F2101-19.

49. The system of embodiment 48, wherein the gas inlet has a bacterial filter.

50. The system of embodiment 48, wherein the flexible, microporous polymer body of the first membrane has a bacterial filtration efficiency of 99.5% or greater

51. The system of embodiment 50, wherein the gas inlet has a bacterial filter.

52. The system of embodiment 50, wherein the introduced gas is CO₂.

53. The system of embodiment 50, wherein the device is arranged to attach to the treatment site.

54. The system of embodiment 50, further comprising a pressure relief valve and wherein introduced gas in the interior chamber of the device can be diverted from the interior chamber and through the pressure relief valve to maintain a desired gas pressure along the treatment site.

55. The system of embodiment 50, further comprising a flush line port and wherein introduced gas in the interior chamber of the device can be diverted from the interior chamber and through the flush line port to flush the introduced gas through the device.

56. The system of embodiment 50, further comprising shapeable structures positioned throughout the device and arranged to conform the device around the treatment site.

57. The system of embodiment 49, wherein the microporous polymer body of the first membrane has a bacterial filtration efficiency in the range of 99.0% to 99.9%.

58. A method for delivering a gas to a treatment site comprising:
providing a device arranged to deliver gas to a treatment site, the device being connectable to a gas source, the device comprising: (i) a first membrane comprising a flexible, microporous polymer body having a bacterial filtration efficiency of 99.9% or greater and (ii) a second membrane comprising a flexible polymer body, wherein the first and second membranes each have outer edge portions and the outer edge portions of the second membrane are bonded to the outer edge portions of the first membrane to form an interior chamber of the device;
positioning and attaching the device to the treatment site;
connecting the gas inlet of the device to the gas source;
supplying the gas to the interior chamber of the device through the gas inlet, the interior chamber of the device confining the supplied gas such that the supplied gas flows out through pores of the first membrane of the device and diffuses the supplied gas into the treatment site,
wherein the supplied gas maintains a gas atmosphere along the treatment site.

59. The method of embodiment 58, wherein the bacterial filtration efficiency of 99.9% or greater of the flexible, microporous polymer body of the first membrane of the device is as determined in accordance with ASTM F2101-19.

60. The method of embodiment 59, wherein the supplied gas is CO₂.

61. The method of embodiment 59, wherein the device is attached to the treatment site by an adhesive positioned on the first membrane.

62. The method of embodiment 59, wherein the device is attached to the treatment site by an adhesive positioned on the second membrane.

63. The method of embodiment 59 wherein the device has edges and wherein the device has a shape formed from the edges of the device, the shape being rectangular, square, triangular, oval, trapezoidal or circular.

64. The method of embodiment 59, wherein the flexible, microporous polymer body of the first membrane of the device is hydrophobic.

65. The method of embodiment 59, wherein the flexible, microporous polymer body of the first membrane of the device is made of polytetrafluoroethylene.

66. The method of embodiment 59, wherein the flexible polymer body of the second membrane of the device is nonporous.

67. The method of embodiment 59, wherein the device further comprises a pressure relief valve and wherein supplied gas in the interior chamber of the device can be diverted from the interior chamber and through the pressure relief valve to maintain a desired gas pressure along the treatment site.

68. The method of embodiment 59, wherein the device further comprises a flush line port and wherein supplied gas in the interior chamber of the device can be diverted from the interior chamber and through the flush line port to flush the supplied gas through the device.

69. The method of embodiment 59, wherein the flexible, microporous polymer body of the first membrane of the device is coated with an antibacterial substance and a medicated substance.

70. The method of embodiment 59, wherein the device further comprises further an open cell sponge support material, the open cell sponge support material being positioned in the interior chamber in direct contact with the flexible, microporous polymer body of the first membrane.

71. The method of embodiment 59, wherein the device further comprises shapeable structures positioned throughout the device, the shapeable structures arranged to shape and conform the device around the treatment site.

72. The method of embodiment 59, wherein the treatment site is a wound.

73. The method of embodiment 59, wherein the treatment site is a surgical site.

74. A method for delivering a gas to a treatment site comprising:
providing a device arranged to deliver gas to a treatment site, the device being connectable to a gas source, the device comprising: (i) a first membrane comprising a flexible, microporous polymer body having a bacterial filtration efficiency of 99.0% or greater and (ii) a second membrane comprising a flexible polymer body, wherein the first and second membranes each have outer edge portions and the outer edge portions of the second membrane are bonded to the outer edge portions of the first membrane to form an interior chamber of the device;
positioning and attaching the device to the treatment site;
connecting the gas inlet of the device to the gas source;
supplying the gas to the interior chamber of the device through the gas inlet, the interior chamber of the device confining the supplied gas such that the supplied gas flows out through pores of the first membrane of the device and diffuses the supplied gas into the treatment site,
wherein the supplied gas maintains a gas atmosphere along the treatment site.

75. The method of embodiment 74, wherein the bacterial filtration efficiency of 99.0% or greater of the flexible, microporous polymer body of the first membrane of the device is as determined in accordance with ASTM F2101-19.

76. The method of embodiment 75, wherein the gas inlet has a bacterial filter.

77. The method of embodiment 75, wherein the flexible, microporous polymer body of the first membrane has a bacterial filtration efficiency of 99.5% or greater

78. The method of embodiment 77, wherein the gas inlet has a bacterial filter.

79. The method of embodiment 76, wherein the supplied gas is CO₂.

80. The method of embodiment 76, wherein the device is attached to the treatment site by an adhesive positioned on the first membrane.

81. The method of embodiment 76, wherein the device is attached to the treatment site by an adhesive positioned on the second membrane.

82. The method of embodiment 76, wherein the device further comprises a pressure relief valve and wherein supplied gas in the interior chamber of the device can be diverted from the interior chamber and through the pressure relief valve to maintain a desired gas pressure along the treatment site.

83. The method of embodiment 76, wherein the device further comprises a flush line port and wherein supplied gas in the interior chamber of the device can be diverted from the interior chamber and through the flush line port to flush the supplied gas through the device.

84. The method of embodiment 76, wherein the device further comprises shapeable structures positioned throughout the device, the shapeable structures arranged to shape and conform the device around the treatment site. 85. The method of embodiment 76, wherein the microporous polymer body of the first membrane has a bacterial filtration efficiency in the range of 99.0% to 99.9%.

86. A device arranged to deliver a gas to a treatment site, the device being connectable to a gas source, the device comprising: (i) a first membrane comprising a flexible, microporous polymer body having a pore size of 0.2 μm or less and (ii) a second membrane comprising a flexible polymer body, the second membrane having a pore size smaller than the first membrane, wherein the first and second membranes each have outer edge portions and the outer edge portions of the second membrane are bonded to the outer edge portions of the first membrane to form an interior chamber of the device, the interior chamber of the device confining introduced gas to the interior chamber such that the introduced gas flows out through the pores of the first membrane, the gas being introduced into the chamber through a gas inlet of the device, the gas inlet being connectable to the gas source, wherein the flexible, microporous polymer body of the first membrane is arranged to diffuse the introduced gas into the treatment site and wherein the introduced gas maintains a gas atmosphere along the treatment site.

87. The device of embodiment 86, wherein the introduced gas is CO₂.

88. The device of embodiment 86, wherein the flexible, microporous polymer body of the first membrane is hydrophobic.

89. The device of embodiment 86, further comprising a pressure relief valve and wherein introduced gas in the interior chamber of the device can be diverted from the interior chamber and through the pressure relief valve to maintain a desired gas pressure along the treatment site.

90. The device of embodiment 86, further comprising a flush line port and wherein introduced gas in the interior chamber of the device can be diverted from the interior chamber and through the flush line port to flush the introduced gas through the device.

91. The device of embodiment 86, further comprising an open cell sponge support material, the open cell sponge support material being positioned in the interior chamber in direct contact with the flexible, microporous polymer body of the first membrane.

92. The device of embodiment 86, further comprising shapeable structures positioned throughout the device and arranged to conform the device around the treatment site.

93. A device arranged to deliver a gas to a treatment site, the device being connectable to a gas source, the device comprising: (i) a first membrane comprising a flexible, microporous polymer body having a pore size of 0.2 μm or greater and (ii) a second membrane comprising a flexible polymer body, the second membrane having a pore size smaller than the first membrane, wherein the first and second membranes each have outer edge portions and the outer edge portions of the second membrane are bonded to the outer edge portions of the first membrane to form an interior chamber of the device, the interior chamber of the device confining introduced gas to the interior chamber such that the introduced gas can only flow out through the pores of the first membrane, the gas being introduced into the chamber through a gas inlet of the device, the gas inlet being connectable to the gas source, wherein the flexible, microporous polymer body of the first membrane is arranged to diffuse the introduced gas into the treatment site and wherein the introduced gas maintains a gas atmosphere along the treatment site.

94. The device of embodiment 93, wherein the gas inlet has a bacterial filter.

95. The device of embodiment 94, wherein the introduced gas is CO₂.

96. The method of embodiment 94, wherein the microporous polymer body of the first membrane has a pore size in the range of 0.2 μm to 0.4 μm.

97. A system comprising a gas source and a device, the device being arranged to deliver a gas to a treatment site, the device being connectable to a gas source, the device comprising: (i) a first membrane comprising a flexible, microporous polymer body having a pore size of 0.2 μm or less and (ii) a second membrane comprising a flexible polymer body, the second membrane having a pore size smaller than the first membrane, wherein the first and second membranes each have outer edge portions and the outer edge portions of the second membrane are bonded to the outer edge portions of the first membrane to form an interior chamber of the device, the interior chamber of the device confining introduced gas to the interior chamber such that the introduced gas flows out through the pores of the first membrane, the gas being introduced into the chamber through a gas inlet of the device, the gas inlet being connectable to the gas source, wherein the flexible, microporous polymer body of the first membrane is arranged to diffuse the introduced gas into the treatment site and wherein the introduced gas maintains a gas atmosphere along the treatment site.

98. The system of embodiment 97, wherein the introduced gas is CO₂.

99. The system of embodiment 97, wherein the flexible, microporous polymer body of the first membrane is made of polytetrafluoroethylene.

100. The system of embodiment 99, wherein the flexible polymer body of the second membrane is nonporous.

101. A system comprising a gas source and a device, the device being arranged to deliver a gas to a treatment site, the device being connectable to a gas source, the device comprising: (i) a first membrane comprising a flexible, microporous polymer body having a pore size of 0.2 μm or greater and (ii) a second membrane comprising a flexible polymer body, the second membrane having a pore size smaller than the first membrane, wherein the first and second membranes each have outer edge portions and the outer edge portions of the second membrane are bonded to the outer edge portions of the first membrane to form an interior chamber of the device, the interior chamber of the device confining introduced gas to the interior chamber such that the introduced gas flows out through the pores of the first membrane, the gas being introduced into the chamber through a gas inlet of the device, the gas inlet being connectable to the gas source, wherein the flexible, microporous polymer body of the first membrane is arranged to diffuse the introduced gas into the treatment site and wherein the introduced gas maintains a gas atmosphere along the treatment site.

102. The system of embodiment 101, wherein the gas inlet has a bacterial filter.

103. The system of embodiment 102, wherein the introduced gas is CO₂.

104. A method for delivering a gas to a treatment site comprising:
providing a device arranged to deliver gas to a treatment site, the device being connectable to a gas source, the device comprising: (i) a first membrane comprising a flexible, microporous polymer body having a pore size of 0.2 μm or less and (ii) a second membrane comprising a flexible polymer body, the second membrane having a pore size smaller than the first membrane, wherein the first and second membranes each have outer edge portions and the outer edge portions of the second membrane are bonded to the outer edge portions of the first membrane to form an interior chamber of the device;
positioning and attaching the device to the treatment site;
connecting the gas inlet of the device to the gas source;
supplying the gas to the interior chamber of the device through the gas inlet, the interior chamber of the device confining the supplied gas such that the supplied gas flows out through the pores of the first membrane of the device and diffuses the supplied gas into the treatment site,
wherein the supplied gas maintains a gas atmosphere along the treatment site.

105. The method of embodiment 104, wherein the supplied gas is CO₂.

106. A method for delivering a gas to a treatment site comprising:
providing a device arranged to deliver gas to a treatment site, the device being connectable to a gas source, the device comprising: (i) a first membrane comprising a flexible, microporous polymer body having a pore size of 0.2 μm or greater and (ii) a second membrane comprising a flexible polymer body, the second membrane having a pore size smaller than the first membrane, wherein the first and second membranes each have outer edge portions and the outer edge portions of the second membrane are bonded to the outer edge portions of the first membrane to form an interior chamber of the device;
positioning and attaching the device to the treatment site;
connecting the gas inlet of the device to the gas source;
supplying the gas to the interior chamber of the device through the gas inlet, the interior chamber of the device confining the supplied gas such that the supplied gas flows out through the pores of the first membrane of the device and diffuses the supplied gas into the treatment site,
wherein the supplied gas maintains a gas atmosphere along the treatment site.

107. The system of embodiment 106, wherein the gas inlet has a bacterial filter.

108. The method of embodiment 107, wherein the supplied gas is CO₂.

109. The method of embodiment 108, wherein the microporous polymer body of the first membrane of the device has a pore size in the range of 0.2 μm to 0.4 μm.

110. The method of embodiment 106, wherein the flexible polymer body of the second membrane is nonporous.

111. The device of embodiment 9, wherein the flexible polymer body of the second membrane has a bacterial efficiency greater than the bacterial efficiency of the first membrane.

112. The device of embodiment 18, wherein the flexible polymer body of the second membrane is nonporous.

113. The device of embodiment 18, wherein the flexible polymer body of the second membrane has a bacterial efficiency greater than the bacterial efficiency of the first membrane.

114. The system of embodiment 32, wherein the flexible polymer body of the second membrane is nonporous.

115. The system of embodiment 32, wherein the flexible polymer body of the second membrane has a bacterial efficiency greater than the bacterial efficiency of the first membrane.

116. The system of embodiment 47, wherein the flexible polymer body of the second membrane is nonporous.

117. The system of embodiment 47, wherein the flexible polymer body of the second membrane has a bacterial efficiency greater than the bacterial efficiency of the first membrane.

118. The method of embodiment 58, wherein the flexible polymer body of the second membrane has a bacterial efficiency greater than the bacterial efficiency of the first membrane.

119. The method of embodiment 74, wherein the flexible polymer body of the second membrane is nonporous.

120. The method of embodiment 74, wherein the flexible polymer body of the second membrane has a bacterial efficiency greater than the bacterial efficiency of the first membrane.

121. The device of embodiment 86, wherein the flexible polymer body of the second membrane is nonporous.

122. The device of embodiment 93, wherein the flexible polymer body of the second membrane is nonporous.

123. The system of embodiment 101, wherein the flexible polymer body of the second membrane is nonporous.

124. The method of embodiment 104, wherein the flexible polymer body of the second membrane is nonporous.

What is claimed is:

1. A device arranged to deliver a gas to a treatment site, the device being connectable to a gas source, the device comprising:
(i) a first membrane comprising a flexible, microporous polymer body, wherein the flexible, microporous polymer body has one of the following features:
(a) a bacterial filtration efficiency of 99.9% or greater and/or a pore size of 0.2 μm or less, or
(b) a bacterial filtration efficiency of 99.0% or greater, and/or a pore size of 0.2 μm or greater, and
(ii) a second membrane comprising a flexible polymer body,
wherein the first and second membranes each have outer edge portions and the outer edge portions of the second membrane are bonded to the outer edge portions of the first membrane to form an interior chamber of the device, the interior chamber of the device confining introduced gas to the interior chamber such that the introduced gas flows out through pores of the first membrane, the gas being introduced into the chamber through a gas inlet of the device, the gas inlet being connectable to the gas source, wherein the flexible, microporous polymer body of the first membrane is arranged to diffuse the introduced gas into the treatment site and wherein the introduced gas maintains a gas atmosphere along the treatment site.

2. The device according to claim 1, wherein the bacterial filtration efficiency is determined in accordance with ASTM F2101-19.

3. The device according to claim 1, wherein the introduced gas is CO₂.

4. The device according to claim 1, wherein the device is arranged to attach to the treatment site.

5. The device according to claim 1, wherein the device is attached to the treatment site by an adhesive positioned on the first membrane or on the second membrane.

6. The device according to claim 1, wherein the device has edges and wherein the device has a shape formed from the edges of the device, the shape being rectangular, square, triangular, oval, trapezoidal or circular.

7. The device according to claim 1, wherein the flexible, microporous polymer body of the first membrane has at least one of the following features:
it is hydrophobic,
it is made of polytetrafluoroethylene, and
it is coated with an antibacterial substance and a medicated substance.

8. The device according to claim 1, wherein the flexible polymer body of the second membrane has at least one of the following features:
it is nonporous,
it has a bacterial efficiency greater than the bacterial efficiency of the first membrane; and
it is made of polytetrafluoroethylene.

9. The device according to claim 1, wherein the device further comprises at least one of:
a pressure relief valve, so that the introduced gas in the interior chamber of the device can be diverted from the interior chamber and through the pressure relief valve to maintain a desired gas pressure along the treatment site;
a flush line port, so that the introduced gas in the interior chamber of the device can be diverted from the interior chamber and through the flush line port to flush the introduced gas through the device;
an open cell sponge support material positioned in the interior chamber in direct contact with the flexible, microporous polymer body of the first membrane; and
shapeable structures positioned throughout the device and arranged to conform the device around the treatment site.

10. The device according to claim 1, wherein the treatment site is a wound or a surgical site.

11. The device according to claim 1, wherein, when the flexible, microporous polymer body of the first membrane has a bacterial filtration efficiency of 99.0% or greater and/or a pore size of 0.2 μm or greater, the gas inlet has a bacterial filter.

12. The device according to claim 1, wherein, when the flexible, microporous polymer body of the first membrane has a bacterial filtration efficiency of 99.0% or greater and/or a pore size of 0.2 μm or greater, the flexible, microporous polymer body of the first membrane has at least one of the following features:
a bacterial filtration efficiency of 99.5% or greater,
a bacterial filtration efficiency in the range of 99.0% to 99.9%, and
a pore size in the range of 0.2 μm to 0.4 μm.

13. A system comprising a gas source and a device according to claim 1.

14. A method for delivering a gas to a treatment site comprising:
providing a device according to claim 1, the device being connectable to a gas source;
positioning and attaching the device to the treatment site;
connecting a gas inlet of the device to the gas source;
supplying a gas to an interior chamber of the device through the gas inlet, the interior chamber of the device confining the supplied gas such that the supplied gas flows out through pores of a first membrane of the device and diffuses the supplied gas into the treatment site,
wherein the supplied gas maintains a gas atmosphere along the treatment site.

15. The method according to claim 13, wherein the supplied gas is CO₂.

16. The method according to claim 13, wherein the device is attached to the treatment site by an adhesive positioned on the first membrane or on the second membrane.
